(19) United States
(12) Patent Application Publication
(10) Pub. No.: US 2025/0325567 A1
(43) Pub. Date: Oct. 23, 2025

(54) COADMINISTRATION OF POLYCANNABINOID WITH AMINE/AMIDE ANESTHETICS TO ENHANCE EFFICACY AGAINST PAIN

(71) Applicant: University of Connecticut, Farmington, CT (US)

(72) Inventors: Gregory A. Sotzing, Storrs, CT (US); Pragati Rout, Storrs, CT (US); Lakshmi S. Nair, New Britain, CT (US); Erick Orozco, New Milford, CT (US)

(21) Appl. No.: 19/183,058

(22) Filed: Apr. 18, 2025

Related U.S. Application Data
(60) Provisional application No. 63/636,513, filed on Apr. 19, 2024.

(51) Int. Cl. A61K 31/00; A61K 9/16; A61K 31/445; A61P 29/00

(52) U.S. Cl. CPC A61K 31/658; A61K 9/1647; A61K 31/445; A61P 29/00

(57) ABSTRACT

Microsphere compositions comprising a cannabinoid compound and a biodegradable polymer are disclosed. Formulations comprising the microsphere compositions and an amine/amide anesthetic, and uses of the formulations for the treatment of pain are further described.

### CROSS-REFERENCE TO RELATED APPLICATIONS

This disclosure claims the benefit of U.S. Provisional Application No. 63/636,513, filed Apr. 19, 2024, the contents of which are hereby incorporated by reference in its entirety for all purposes.

### TECHNICAL FIELD

The invention disclosed herein relates to pain relief, and in particular to synergistic compositions for enhancement of anesthetic effects.

### BACKGROUND

Osteoarthritis (OA) stands as one of the most devastating conditions, imposing a substantial personal and economic burden. The impact of this condition is profound due to its high disability rates, morbidity, treatment costs, and its high risk of mortality. While there are ample treatments to manage pain and symptoms associated with OA, none of them has proven entirely effective or has reversed exiting effects. According to Medical News Today, it is projected that globally around 500 million people or 7% of the global population is impacted by OA. More than 32 million people are suffering from OA in USA. The most concerning fact is that among the victims of OA only 60% are expected to be symptomatic. Rising concerns for patients only detecting it at later stages.

Multiple factors like age, obesity, injury, stress on joints, muscle weakness, joint malalignment, and genetics contribute to the onset and progression of OA. It results from the breakdown of joint cartilage and the bone beneath it, thereby increasing the friction between two joints. One of the most important factors in OA progression is joint inflammation. Both proinflammatory and anti-inflammatory factors, as well as extracellular matrix degradation enzymes (matrix metalloproteinases (MMPs) play an important role in disease development. This condition can be managed either by pharmacological or non-pharmacological methods. Non-pharmacological methods involve muscle strengthening, joint movement by physiotherapy, acupuncture, herbal therapy, weight loss etc., whereas pharmacological methods involve pain killers, nonsteroidal anti-inflammatory drugs (NSAIDS, e.g., ibuprofen, aspirin, diclofenac, naproxen), opioids, and corticosteroids administered by various routes (parenteral, oral, topical). The severe side effects and short-term efficacy of the drugs renders them unsuitable and inconvenient for many patients.

To combat this long term, and chronic disease the alternative methods practiced are joint surgery or joint replacement, which are so expensive that it is not affordable by many patients. This unmet need underscores the urgent requirement for innovative therapeutic approaches which are non-severe and efficient.

Therapeutic pain relief is vitally important in the medicinal arts. Unfortunately, many anesthetics have detrimental side effects. Phyto cannabinoids are long known for their therapeutic effect on humans and animals. Their potential to manage pain and inflammation is not hidden from scientists and pharmacists. However, despite these known benefits, challenges such as low bioavailability and unclear modes of action within the body have hindered their widespread use and marketability.

What are needed are improved methods, apparatus, and compositions of matter for pain relief.

### SUMMARY

Disclosed herein is a formulation, comprising: a microsphere composition; and an amine/amide anesthetic; wherein the microsphere composition comprises a cannabinoid according to formula (I) or (II)

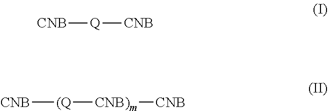

wherein each CNB individually is a cannabinoid moiety; Q is a linking group attached to each cannabinoid moiety independently through an amine, an ether, a thioether, an ester, or an amido group; and m is 1-10; and a biodegradable polymer.

Further disclosed herein is a method for the treatment of pain, comprising: co-administering to a subject in need thereof, a microsphere composition; and an amine/amide anesthetic; wherein the microsphere composition comprises a cannabinoid according to formula (I) or (II)

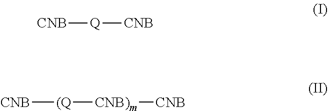

wherein each CNB individually is a cannabinoid moiety; Q is a linking group attached to each cannabinoid moiety independently through an amine, an ether, a thioether, an ester, or an amido group; and m is 1-10; and a biodegradable polymer.

Also disclosed herein is a microsphere composition, comprising: a cannabinoid according to formula (I) or (II)

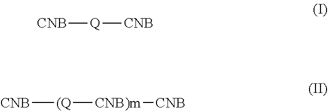

wherein each CNB individually is a cannabinoid moiety; Q is a linking group attached to each cannabinoid moiety independently through an amine, an ether, a thioether, an ester, or an amido group; and m is 1-10; and a biodegradable polymer.

### BRIEF DESCRIPTION OF THE DRAWINGS

The features and advantages of the invention are apparent from the following description taken in conjunction with the accompanying drawings in which:

FIG. 1 illustrates the chemical structure of CBD-adipate dimer.

FIG. 2 illustrates the chemical structure of Ropivacaine.

FIG. 3 illustrates examples of anesthetic compounds in the amine-amide class of anesthetics having structures similar to Ropivacaine; these compounds are used for pain management of osteoarthritis.

FIG. 4 Dose dependent cytotoxicity of Dicannabidiol (diCBD) adipate and AM1241 towards F-11 (neuronal) cells after 24 hour incubation.

FIG. 5 Anti-inflammatory efficacy of Dicannabidiol (diCBD) adipate and AM1241 towards inflamed bone marrow macrophages.

FIG. 6 Preparation process of Dicannabidiol adipate PLGA microspheres.

FIGS. 7A-7C Representative SEM images of Dicannabidiol (diCBD) adipate loaded microspheres prepared using serially increasing diCBD to PLGA ratios: FIG. 7A) 1:3 diCBD:PLGA; FIG. 7B) 1:2 diCBD:PLGA; FIG. 7C) 1:1 diCBD:PLGA.

FIG. 7D Size analysis quantification of diCBD microspheres prepared using serially increasing diCBD to PLGA ratios.

FIG. 7E diCBD % load quantification of microspheres with serially increasing diCBD to PLGA ratios.

FIG. 8 In vitro release profile of Dicannabidiol adipate from PLGA microsphere delivery system shows moderate and controlled release of dicannabidiol adipate for at least 8 weeks in vitro.

FIG. 9 Visual representation of in vivo experimental timeline for a rat osteoarthritis model.

FIG. 10A-10B Analgesic efficacy of Dicannabidiol adipate microsphere treatments as measured by changes to mechanical allodynia. FIG. 10A) Dicannabidiol adipate microspheres suspended in HA solution. FIG. 10B) diCBD adipate microspheres suspended in an HA solution with Ropivacaine.

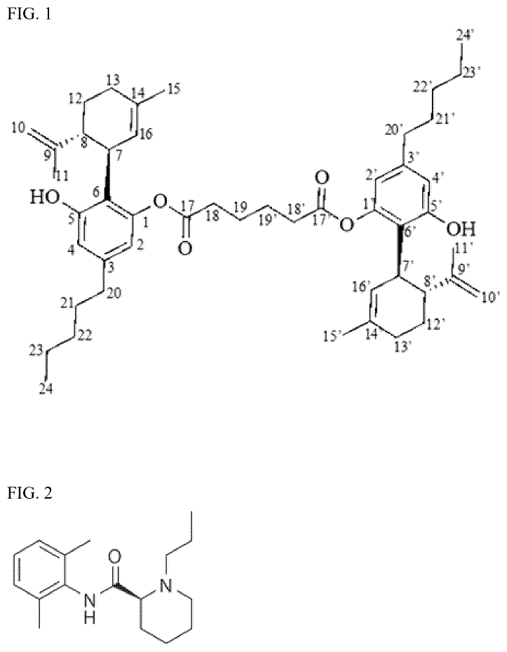

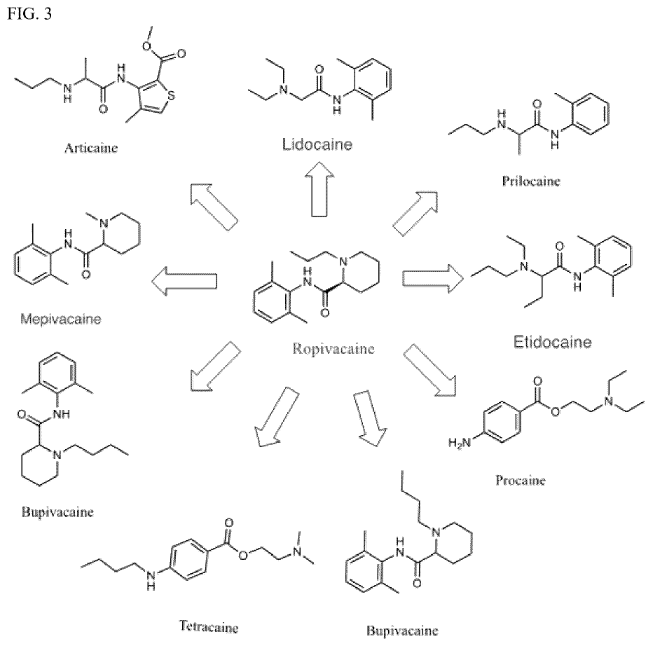

### DETAILED DESCRIPTION

Phyto cannabinoids are long known for their therapeutic effect on humans and animals. It has been used to treat pain for thousands of years in its herbal form. The overall effects of herbal cannabis represent the collective activity of Tetrahydrocannabinol (THC), Cannabidiol (CBD), and several trace cannabinoids. Cannabidiol (CBD) is a molecule found in herbal cannabis in large amounts. Although CBD does not produce psychotropic effects, it has been shown to produce a variety of pharmacological effects. In the recent advancement in CBD research and its bio-interaction mechanism, it has been found that CBD converts to THC in room temperature by cyclization of free hydroxyl group with isopropyl group which are present in the CBD molecule. However, the same effect is not seen in non-therapeutic polymeric form of CBD like polyCBD adipate. This led to the concept of making dimers and oligomers of cannabinoids through diacid linkers which is further discussed herein.

In certain embodiments, the cannabinoid dimer or oligomer has the following structure of Formula (I):

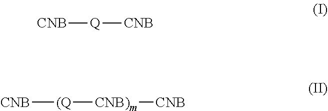

wherein each CNB individually is a cannabinoid moiety; Q is a linking group attached to each cannabinoid moiety independently through an amine, an ether, a thioether, an ester, or an amido group, specifically an ester group; and m is 1, 2, 3, 4, 5, 6, 7, 8, 9, or 10.

The cannabinoid moiety CNB of formulas (I) and (II) can be derived from any natural or synthetic cannabinoid. Cannabinoids can be classified by making subgroups of the main molecule. For example, cannabinoids having structural similarity with CBD, THC or Cannabigerol (CBG) and other miscellaneous cannabinoids.

### Cannabidiol and Analogs

1

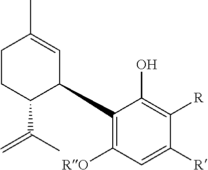

| Sl.N | Molecule Name | R | R′ | R″ |
|---|---|---|---|---|
| 1a | Cannabidiol | H | C₅H₁₁ | H |
| 1b | Cannabidiolic acid | COOH | C₅H₁₁ | H |
| 1c | Cannabidiol monomethylether | H | C₅H₁₁ | CH₃ |
| 1d | Cannabidibutol | H | C₄H₉ | H |
| 1e | Cannabidivarine | H | C₃H₇ | H |
| 1f | Cannabidivarinic acid | COOH | C₃H₇ | H |
| 1g | Cannabidiorcol | H | CH₃ | H |
| 1h | Cannabidihexol | H | C₆H₁₃ | H |
| 1i | Cannabidiphorol | H | C₇H₁₅ | H |

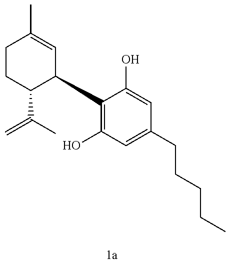 [SMILES: C=C(C)[C@@H]1CCC(C)=C[C@H]1c1c(O)cc(CCCCC)cc1O]

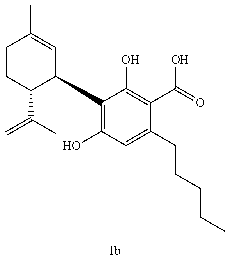 [SMILES: C=C(C)[C@@H]1CCC(C)=C[C@H]1c1c(O)cc(CCCCC)c(C(=O)O)c1O]

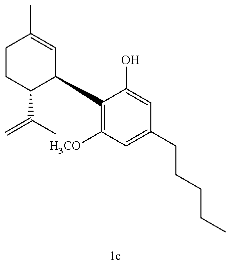 [SMILES: C=C(C)[C@@H]1CCC(C)=C[C@H]1c1c(O)cc(CCCCC)cc1OC]

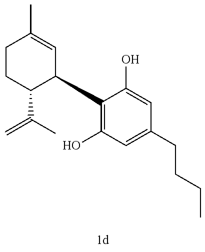 [SMILES: C=C(C)[C@@H]1CCC(C)=C[C@H]1c1c(O)cc(CCCC)cc1O]

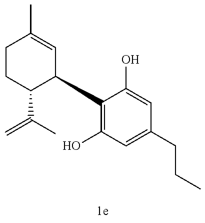 [SMILES: C=C(C)[C@@H]1CCC(C)=C[C@H]1c1c(O)cc(CCC)cc1O]

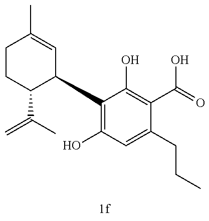 [SMILES: C=C(C)[C@@H]1CCC(C)=C[C@H]1c1c(O)cc(CCC)c(C(=O)O)c1O]

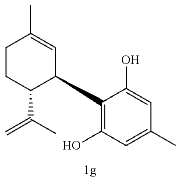 [SMILES: C=C(C)[C@@H]1CCC(C)=C[C@H]1c1c(O)cc(C)cc1O]

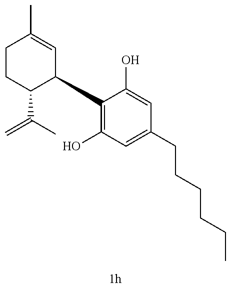 [SMILES: C=C(C)[C@@H]1CCC(C)=C[C@H]1c1c(O)cc(CCCCCC)cc1O]

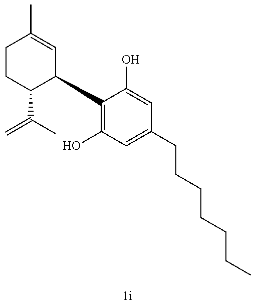 [SMILES: C=C(C)[C@@H]1CCC(C)=C[C@H]1c1c(O)cc(CCCCCCC)cc1O]

### Cannabigerol and Analogs

2

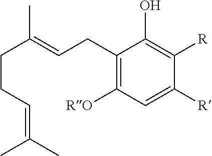

| Sl.N | Molecule Name | R | R′ | R″ |
|---|---|---|---|---|
| 2a | Cannabigerol | H | C5H11 | H |
| 2b | Cannabigerolic acid | COOH | C5H11 | H |
| 2c | Cannabigerol monomethylether | H | C5H11 | CH3 |
| 2d | Monomethylether Cannabigerolic acid | COOH | C5H11 | CH3 |
| 2e | Cannabigerovarine | H | C3H7 | H |
| 2f | Cannabigerovarinic acid | COOH | C3H7 | H |

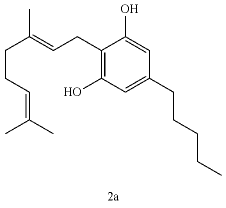 [SMILES: CCCCCc1cc(O)c(C/C=C(\C)CCC=C(C)C)c(O)c1]

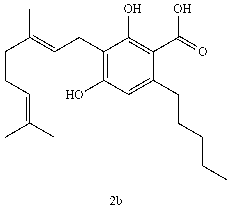 [SMILES: CCCCCc1cc(O)c(C/C=C(\C)CCC=C(C)C)c(O)c1C(=O)O]

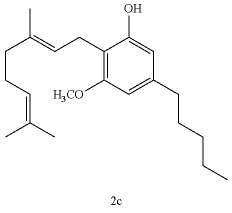 [SMILES: CCCCCc1cc(O)c(C/C=C(\C)CCC=C(C)C)c(OC)c1]

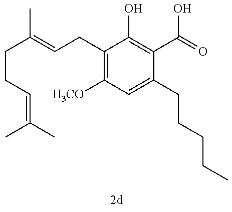 [SMILES: CCCCCc1cc(OC)c(C/C=C(\C)CCC=C(C)C)c(O)c1C(=O)O]

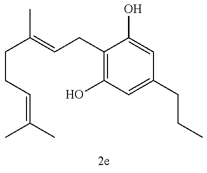 [SMILES: CCCc1cc(O)c(C/C=C(\C)CCC=C(C)C)c(O)c1]

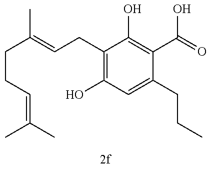 [SMILES: CCCc1cc(O)c(C/C=C(\C)CCC=C(C)C)c(O)c1C(=O)O]

### D9-Tetrahydrocannabinol and Analogs

3

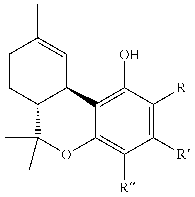

| Sl.No | Molecule Name | R | R′ | R″ |
|---|---|---|---|---|
| 3a | D9-tetrahydrocannabinol | H | C5H11 | H |
| 3b | D9-tetrahydrocannabinolic acid | COOH | C5H11 | H |
| 3c | D9-tetrahydrocannabinolic acid B | H | C5H11 | COOH |
| 3d | D9-tetrahydrocannabinol-C4 | H | C4H9 | H |
| 3e | D9-tetrahydrocannabinolic acid-C4 | COOH | C4H9 | H |
| 3f | D9-tetrahydrocannabivarin | H | C3H7 | H |
| 3g | D9-tetrahydrocannabivarinic acid | COOH | C3H7 | H |
| 3h | D9-tetrahydrocannabiorcol | H | CH3 | H |
| 3i | D9-tetrahydrocannabiorcolic acid | COOH | CH3 | H |
| 3j | D9-tetrahydrocannabinal | CHO | C5H11 | H |
| 3k | D9-tetrahydrocannabiphorol | H | C7H15 | H |
| 31 | D9-tetrahydrocannabihexol | H | C6H13 | H |

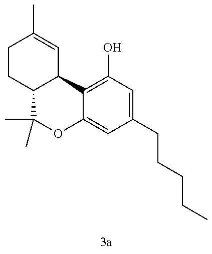 [SMILES: CCCCCc1cc(O)c2c(c1)OC(C)(C)[C@@H]1CCC(C)=C[C@@H]21]

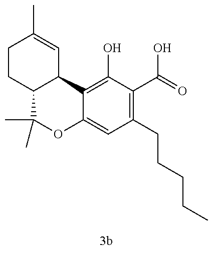 [SMILES: CCCCCc1cc2c(c(O)c1C(=O)O)[C@@H]1C=C(C)CC[C@H]1C(C)(C)O2]

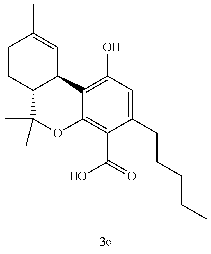 [SMILES: CCCCCc1cc(O)c2c(c1C(=O)O)OC(C)(C)[C@@H]1CCC(C)=C[C@@H]21]

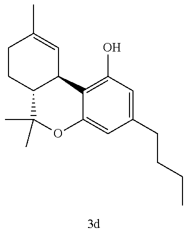 [SMILES: CCCCc1cc(O)c2c(c1)OC(C)(C)[C@@H]1CCC(C)=C[C@@H]21]

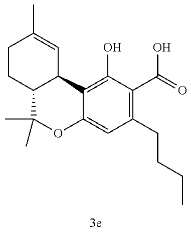 [SMILES: CCCCc1cc2c(c(O)c1C(=O)O)[C@@H]1C=C(C)CC[C@H]1C(C)(C)O2]

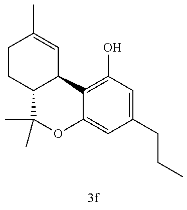 [SMILES: CCCc1cc(O)c2c(c1)OC(C)(C)[C@@H]1CCC(C)=C[C@@H]21]

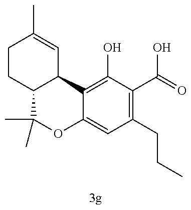 [SMILES: CCCc1cc2c(c(O)c1C(=O)O)[C@@H]1C=C(C)CC[C@H]1C(C)(C)O2]

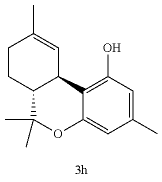

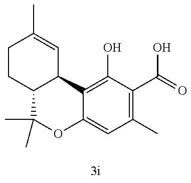 [SMILES: CC1=C[C@H]2c3c(cc(C)c(C(=O)O)c3O)OC(C)(C)[C@@H]2CC1]

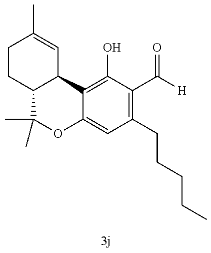 [SMILES: CCCCCc1cc2c(c(O)c1C=O)[C@@H]1C=C(C)CC[C@H]1C(C)(C)O2]

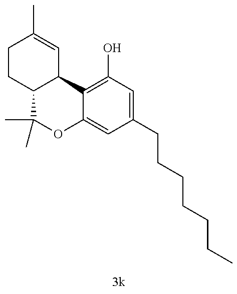 [SMILES: CCCCCCCc1cc(O)c2c(c1)OC(C)(C)[C@@H]1CCC(C)=C[C@@H]21]

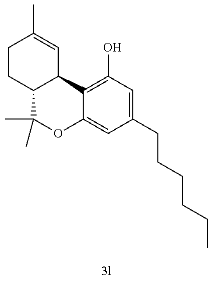 [SMILES: CCCCCCc1cc(O)c2c(c1)OC(C)(C)[C@@H]1CCC(C)=C[C@@H]21]

### D8-Tetrahydrocannabinol and Analogs

4

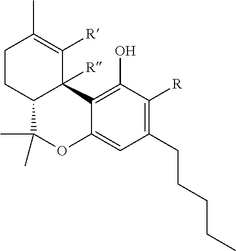

| Sl.No | Molecule Name | R | R′ | R″ |
|---|---|---|---|---|
| 4a | D8-tetrahydrocannabinol | H | H | H |
| 4b | D8-tetrahydrocannabinolic acid | COOH | H | H |
| 4c | 10α-hydroxy-Δ8-tetra-hydrocannabinol | H | α-OH | H |
| 4d | 10β-hydroxy-Δ8-tetra-hydrocannabinol | H | H | β-OH |
| 4e | 10a-α-hydroxy-10-oxo-Δ8- | H | O | OH |
| | tetrahydrocannabinol | | | |

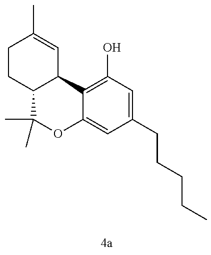 [SMILES: CCCCCc1cc(O)c2c(c1)OC(C)(C)[C@@H]1CCC(C)=C[C@@H]21]

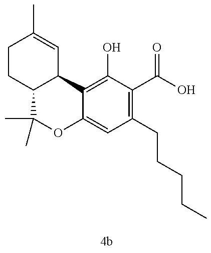 [SMILES: CCCCCc1cc2c(c(O)c1C(=O)O)[C@@H]1C=C(C)CC[C@H]1C(C)(C)O2]

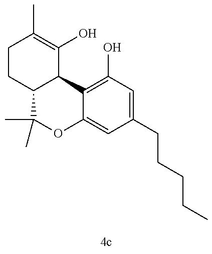 [SMILES: CCCCCc1cc(O)c2c(c1)OC(C)(C)[C@@H]1CCC(C)=C(O)[C@@H]21]

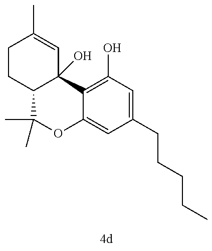 [SMILES: CCCCCc1cc(O)c2c(c1)OC(C)(C)[C@@H]1CCC(C)=C[C@@]21O]

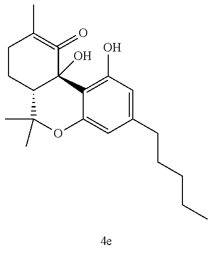

### Other Miscellaneous Cannabinoids Having Monols, Diols and Triols:

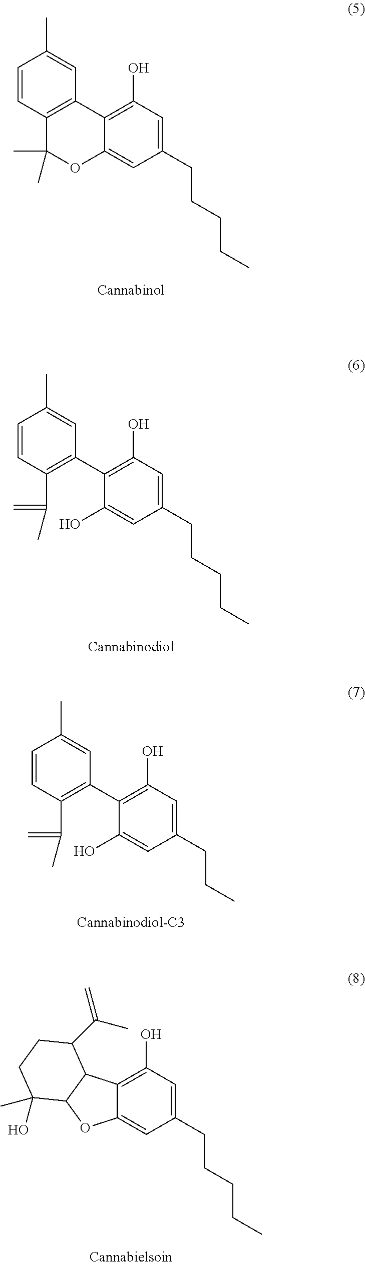

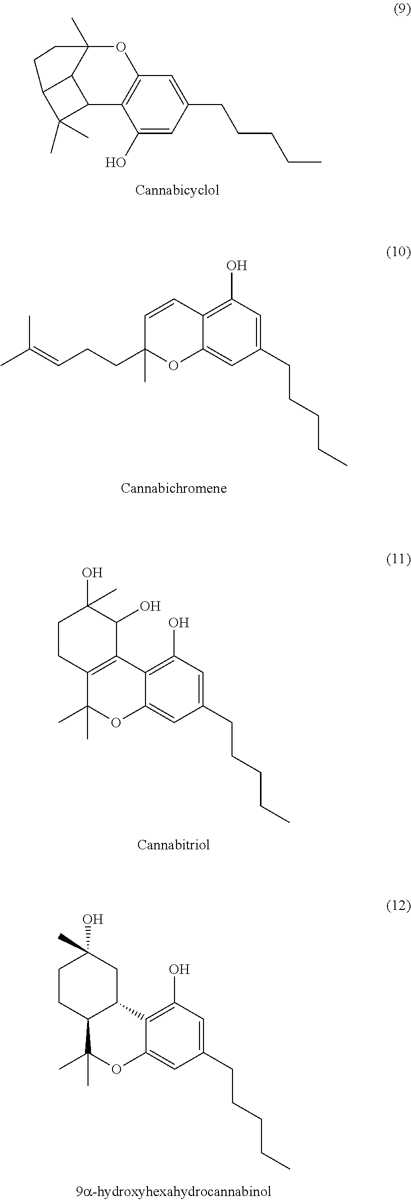

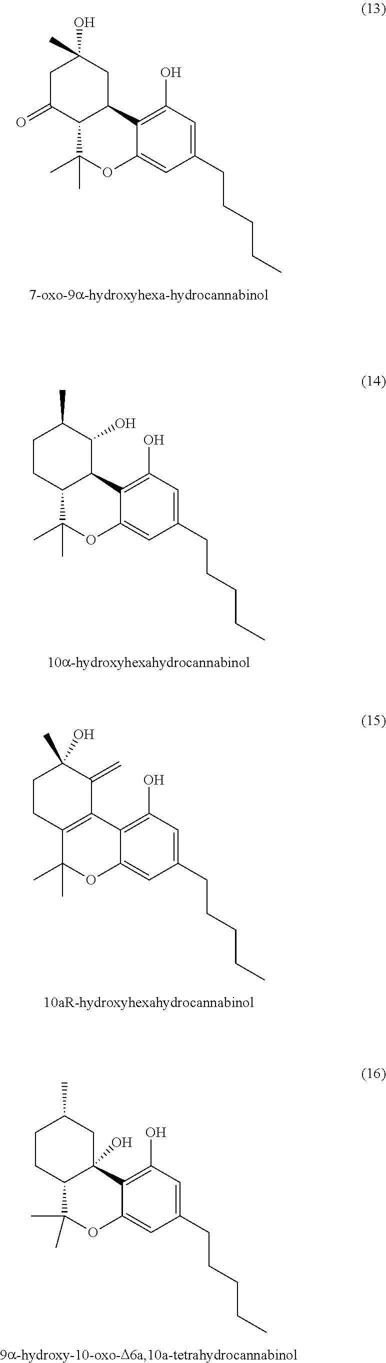

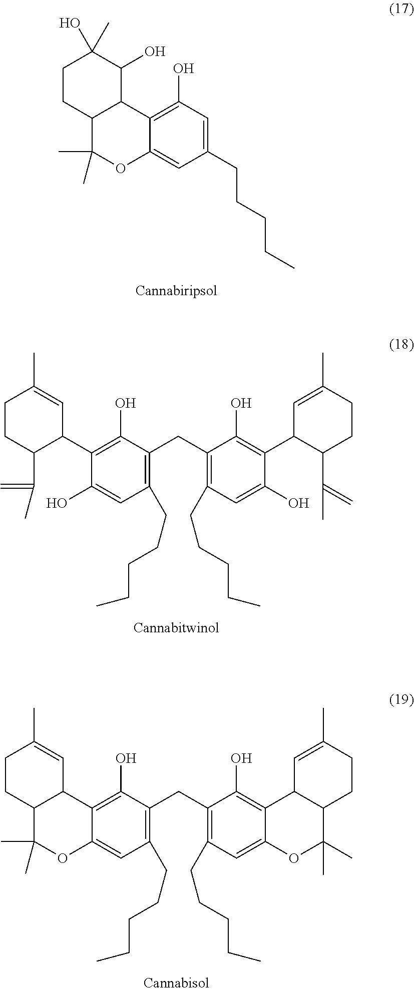

The L linking group of Formula (I) and (II) can be a C₁-C₄₀ alkyl group, specifically C₁-C₃₀ alkyl group, more specifically a C₂-C₁₈ alkyl group, yet more specifically a C₃-C₁₀ alkyl group; an ethylene glycol group (—OCH₂CH₂—), specifically an ethylene glycol group having 1-20 ethylene glycol residues; a propylene glycol group, specifically a propylene glycol group having 1-20 propylene glycol residues; an aryl group; a heteroaryl group; or a heterocyclic group. In an embodiment, L1 linking group of Formula (I) and (II) can be an internally substituted C₂-C₄₀ alkyl group, specifically C₂-C₃₀ alkyl group, more specifically a C₂-C₁₈ alkyl group, yet more specifically a C₃-C₁₀ alkyl group containing 1, 2, or 3 heteroatoms within the carbon chain itself (not at a terminus) wherein the heteroatom is O, S, N, or a combination thereof. For example, the linking group can be an internally substituted C₄ alkyl group having a single O or N within the carbon chain, such as —CH₂CH₂OCH₂CH₂— and —CH₂CH₂NHCH₂CH₂—. In a further example, the linking group can be an internally substituted C₄ alkyl group having 2 or 3 O, S, or N within the carbon chain, such as —CH₂CH₂O(C═O)CH₂CH₂—, —CH₂CH₂(C═O)NHCH₂CH₂—, and —CH₂CH₂NH(C═O)NHCH₂CH₂—.

The phytocannabinoids, trace cannabinoids and their derivatives having free-OH group can be dimerized, e.g., using a diacid linker to synthesize dimers of cannabinoids. Suitable diacid linkers or derivatives thereof include, for example, fumaric acid, glutamic acid, maleic acid, malic acid, terephthalic acid, isophthalic acid, naphthalene-2,6-dicarboxylic acid, oxaloacetic acid, phthalic acid, butanedioic acid, oxalic acid, malonic acid, succinic acid, tartaric acid, glutaric acid, adipic acid, pimelic acid (heptanedioic acid), suberic acid (octanedioic acid), azelaic acid (nonanedioic acid), sebacic acid (decanedioic acid), undecanedioic acid, dodecanedioic acid, pyridine-2,6-dicarboxylic acid, 1H-imidazole-4,5-dicarboxylic acid, furan-2,5-dicarboxylic acid, furan-2,3-dicarboxylic acid, thiophene-2,5-dicarboxylic acid, thiophene-2,3-dicarboxylic acid, quinoline-2,4-dicarboxylic acid, cyclohexane-1,4-dicarboxylic acid, cyclopentane-1,3-dicarboxylic acid, cyclobutane-1,3-dicarboxylic acid, or bicyclo[2.2.2]octane-1,4-dicarboxylic acid, or a bifunctional compound such as

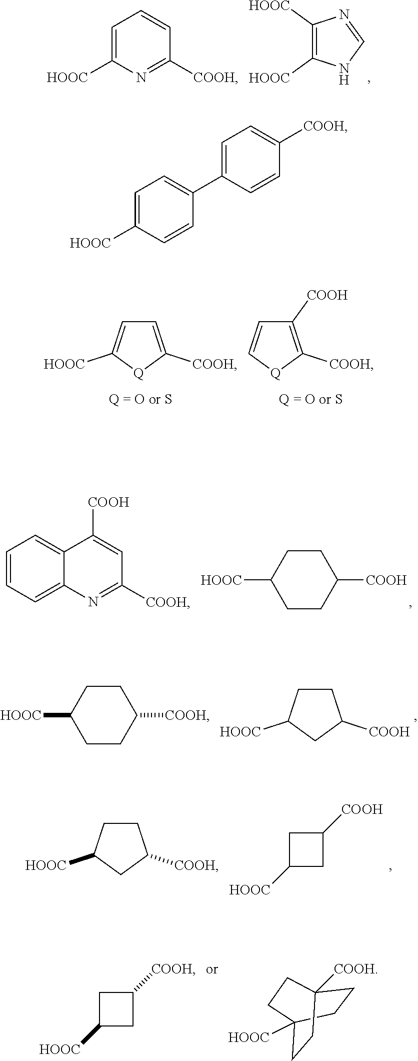

In an embodiment, a cannabinoid dimer is Cannabidiol adipate dimer, also referred to as Dicannabidiol adipate or diCBD adipate.

The cannabinoid dimers and oligomers may be formed by solventless procedures (e.g. melt polymerizations) as well as those employing solvent including combinations of pure monomers if both are liquids including the melting of CBD or other cannabinoid to form a liquid. In an embodiment, the reaction can be carried out in a solvent.

In general cannabinoid dimers and oligomers may be formed by reaction of a cannabinoid comprising a hydroxyl group with a diacid linker comprising carboxylic acids or a derivative thereof, e.g., a diester, a dianhydride, a diacid chloride, and the like. Esterification and transesterification reactions may be employed.

In an embodiment, the cannabinoid moieties of prepared cannabinoid dimers and oligomers can be derivatized to modify the properties of the compounds. For example, the cannabinoid moieties may be derivatized to make sulfonic acid/sulfonate derivatives to increase the compound's water solubility. Optionally, the linking group can also be derivatized to increase water solubility.

### Microsphere Systems

The cannabinoid dimers and oligomers can be prepared into microsphere systems suitable for use in injectable formulations, oral formulations, topical, and the like.

The microsphere systems comprise a cannabinoid dimer or cannabinoid oligomer, and a biodegradable and/or biocompatible polymer. Suitable biodegradable and/or biocompatible polymers can be prepared from caprolactone, glycolic acid, lactic acid, or a combination thereof, and the like, including a polylactic acid, a pol (glycolic acid), a poly(lactic-co-glycolic) acid (PLGA), a polycaprolactone, etc. Still other suitable biodegradable and/or biocompatible polymers can be polycannabinoids. Polycannabinoids may be used to increase cannabinoid dimer or cannabinoid oligomer concentration in the microsphere system from 30 wt % loading to values higher than 50 wt % based upon the ‘like dissolves like’ principle in Organic Chemistry.

The polycannabinoid biodegradable polymer can have the formula:

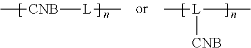

wherein:
CNB is a cannabinoid moiety,
L is a linking group; and
n represents the number of repeat units wherein n is at least 2. In general. the polycannabinoid biodegradable polymers have a number average molecular weight of about 5,000 daltons to about 60,000 daltons, specifically about 6,000 daltons to about 55,000 daltons, more specifically about 7,000 daltons to about 50,000 daltons, yet more specifically about 8,000 daltons to about 50,000 daltons, still more specifically about 9,000 to about 40,000 daltons, and yet more specifically about 10,000 to about 30,000 daltons. Suitable polycannabinoid biodegradable polymers include those described in US Patent Application Publication US2021/0322365A1 to Sotzing.

The cannabinoid dimer can be incorporated in various concentrations within the biodegradable polymer microspheres. This allows for the preparation of microsphere formulations having different doses of cannabinoid dimer, which can be tailored to the application of choice. The weight ratio of cannabinoid dimer/oligomer to biodegradable polymer can be 1:6, 1:5, 1:4, 1:3, 1:2, 1:1 and 2:1.

The microspheres of the microsphere system can be substantially spherical to spherical.

The average size of the microspheres of the microsphere system can be below 40 μm, specifically about 1 to about 20 μm, and more specifically about 4 to about 10 μm in diameter. The particle size may be determined by Scanning electron microscopy (SEM) or other suitable technique.

Compositions with Amine/Amide Anesthetics; Use in Pain Relief

Further disclosed herein are methods, systems, and compositions of matter for pain relief. The cannabinoid dimers and oligomers, and microsphere systems comprising the cannabinoid dimers and oligomers can be combined or co-administered with amine/amide anesthetics to provide synergistic analgesic effect.

Suitable amine/amide anesthetic that can be used in combination with the cannabinoid dimers, oligomers, and microsphere systems include Articaine, Bupivacaine, Etidocaine, Lidocaine, Mepivacaine, Prilocaine, Procaine, Ropivacaine, Tetracaine, and the like. In an embodiment the amine/amide anesthetic is Ropivacaine.

The cannabinoid dimer, cannabinoid oligomer, or microsphere systems thereof, can be combined with the amine/amide anesthetic into a single formulation for administration to a subject in need thereof or co-administered as separate formulations. The combination can provide a synergistic effect with anti-inflammatory molecules. Co-administration allows acute relief and long-term improvement of latent pain. The formulations can be administered by any route, specifically parenterally, orally, or topically. The parenteral administration can be intravenously, subcutaneously, intramuscularly, or locally at a specific site in the subject.

In a study, PLGA nanoparticles loaded with CBD-adipate dimer was co-administered intravenously along with the amine/amide anesthetic Ropivacaine into a rat model of osteoarthritis. Ropivacaine alone provided a duration of the rat being pain free from 2 days while the addition with the CBD-adipate dimer increased the pain free duration to 3 days. Furthermore, as opposed to the pain associated with osteoarthritis returning to a higher pain level, as is normal for local anesthetics, there was less pain after the action of the Ropivacaine, consistent with the CBD-adipate dimer being used alone as the control.

In vitro, CBD-adipate dimer showed minimal dose dependent cell toxicity, and potent anti-inflammatory effect. In vivo, in a rat osteoarthritic model, localized injection of CBD-adipate dimer microsphere system was able to provide significant pain relief for a period of up to 3-4 weeks. The CBD-adipate dimer microsphere formulation has the potential to be a therapeutic treatment option for diseases with localized inflammation and/or pain.

CBD-adipate dimer shows anti-inflammatory and analgesic properties, by itself, increasing its therapeutic potential beyond that of osteoarthritic joint disease. The combination of CBD-adipate dimer with Ropivacaine offers 3 days, pain-free, as opposed to 2 days only using Ropivacaine, and after 3 days the pain level returns to be less than that of the initial pain (unlike Ropivacaine alone where there is more pain once the drug is no longer active) for 3 weeks. Thus, the combination provided improved pain response for several weeks including 50% more acute relief.

The CBD-adipate dimer microsphere system provides prolonged pain relief from a single injection in vivo. The microsphere formulation was designed to 1) provide long lasting pain relief, 2) provide pain relief from a single administration, 3) be minimally invasive, and 4) have low potential of misuse and abuse as it is non-addictive. These characteristics increase the ease of use of this formulation in a clinical, out-patient setting. As this new system provides prolonged pain relief, the patient requires fewer visits to a clinician, reducing both the burden of cost on the patient, and on the health care system. As this formulation provides pain relief from a single administration, the pain relief effects are immediately noticeable increasing patient well-being. The injectable formulation is easy to administer, and by being a locally injectable formulation, the risk of systemic side effects is greatly reduced.

The cannabinoid dimers and oligomers, microsphere systems comprising the cannabinoid dimers and oligomers combined with amine/amide anesthetics for analgesic effect can be administered to a subject in need of treatment. The subject can be a mammal, including humans and animals, including companion animals and livestock.

In an embodiment, the cannabinoid dimer, cannabinoid oligomer, microsphere system comprising the cannabinoid dimer or oligomer, either alone or combined with an amine/amide anesthetic can be used to remediate pain and/or inflammation. The pain may be associated with osteoarthritis, post-operative pain, etc.

Disclosed herein are formulations comprising an effective amount of the cannabinoid dimer, cannabinoid oligomer, or microsphere system comprising the cannabinoid dimer or oligomer, a pharmaceutically acceptable excipient, and optionally further comprising an effective amount of an amine/amide anesthetic. These formulations can be administered by any route of administration, including oral, parenteral (intramuscular, intravenous, subcutaneous, intra-articular, intra-synovial, intraperitoneal, intrasternal, intrathecal, intrahepatic, intralesional and intracranial), or topical routes. The compounds as described herein may be administered in single or divided doses.

In an embodiment, the cannabinoid dimer, cannabinoid oligomer, or amine/amide anesthetic can be in the form of a pharmaceutically acceptable salt, e.g., an acid or base addition salt of compounds as described herein. The acids which are used to prepare the pharmaceutically acceptable acid addition salts are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others. The chemical bases that may be used as reagents to prepare pharmaceutically acceptable base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, exemplary carriers are physiological saline or phosphate buffered saline (PBS).

The compositions as described herein may be orally administered in any orally acceptable dosage form including, but not limited to, capsules, tablets, aqueous suspensions or solutions.

The amount of compound in a composition as described herein that may be combined with the carrier materials to produce a single dosage form will vary depending upon the cannabinoid, the host and the disease or symptom treated, and the particular mode of administration. The compositions can be formulated to contain between about 0.05 milligram to about 750 milligrams or more, specifically about 1 milligram to about 600 milligrams, and more specifically about 10 milligrams to about 500 milligrams of active ingredient, alone or in combination with at least one other compound according to the present disclosure. A particular advantage of the cannabinoid dimers and oligomers described herein is the ability to prepare and administer accurate concentrations of a cannabinoid due to the dimer/oligomer's stability against thermal degradation and stability against unwanted conversion of the target cannabinoid to other cannabinoid compounds.

It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease or condition being treated.

In certain embodiments, the method comprises administering to a subject in need thereof an effective amount of a cannabinoid dimer or oligomer as described herein, optionally including a pharmaceutically acceptable excipient, and further optionally an amine/amide anesthetic to treating or ameliorating a disease, disorder or symptom thereof in a subject, e.g., an animal such as a human. In an embodiment, the method is for the treatment of pain.

In further embodiments, a method for the treatment of pain comprises co-administering to a subject in need thereof, an effective amount of a microsphere composition; and an effective amount of an amine/amide anesthetic; wherein the microsphere composition comprises a biodegradable polymer and a cannabinoid according to formula (I) or (II)

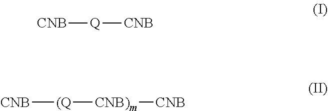

wherein each CNB individually is a cannabinoid moiety; Q is a linking group attached to each cannabinoid moiety independently through an amine, an ether, a thioether, an ester, or an amido group, specifically an ester group; and m is 1-10.

The following examples are merely illustrative of the cannabinoid dimers and oligomers, compositions thereof, and uses thereof disclosed herein and are not intended to limit the scope thereof.

### EXAMPLES

### Example 1. Synthesis of Dicannabidiol Adipate

### Experimental Procedure: (CBD:AC, 1:0.7)

Into a clean, vacuum dried and flame dried round bottom flask was added Cannabidiol (CBD) (10 g, 0.0318 mol), then a mixture of 80 mL of anhydrous methylene chloride (DCM) and 40 mL of pyridine. The mixture was stirred for half an hour at room temperature to attain equilibrium. The solution was then chilled to 5° C. in an ice water bath. Adipoyl chloride (4.07 g, 0.022 mol) was added dropwise over 30 minutes. The reaction mixture was stirred for 4 days. The solution was concentrated under vacuum and proceeded for washing. The mixture was given 3 times deionized (DI) water wash followed by 3 times 10% HCL in water wash and finally 3 times brine wash. The organic layer was collected and then dehydrated by adding MgSO₄ and filtered. The filtrate was concentrated using vacuum and proceeded for column chromatography (Hexane:Ethyl Acetate). The fraction collected from the column was environmentally dried. The brown viscous liquid collected was again proceeded for a second column chromatography (Hexane:EthylAcetate). The fraction collected from the second column was environmentally dried, yielding brownish white crystals. The brownish white crystals collected from column were crushed finely. Then a slurry of 5 mL hexane and the product was prepared. Then proceeded for funnel filtration. The solid was washed with 5 ml hexane and then dried. White crystalline powder of diCBD adipate obtained (2 g). Molecular Weight 739.05; C₄₈H₆₆O₆. M.P 125° C. The structure of diCBD adipate was confirmed by IR spectroscopy; mass spectroscopy; ¹H and ¹³C NMR spectroscopy COSY NMR, HSQC NMR, HMBC NMR, and NOESY NMR (taken on BUKER 500 MHz and 800 MHz NMR spectroscopy instruments).

Dicannabidiol adipate was obtained as a white crystal solid and displayed a protonated molecular ion [M+H]+ at m/z 739.4940 (calcd 739.4940 for C₄₈H₆₆O₆+H) in the positive mode HRMS, corresponding to a molecular formula of C₄₈H₆₆O₆. The ¹³C NMR spectrum, however, displayed only 24 resonances, suggesting the possibility of a dimeric structure. Two sp³ methylene resonance at δ_{C} 24.3 and δ_{C} 34.24 was observed in ¹³C NMR spectra and showed HSQC correlation with two of proton signal δ_{H} 1.85 and δ_{H} 2.58 in the ¹H NMR spectrum respectively. The rest of the NMR spectrum was close to those of CBD except for the resonance of aromatic protons which now show two different singlets i.e δ_{H} 6.41 and δ_{H} 6.57 possibly due to the bonding at hydroxy position the resonance shifted, one OH peak is also missing as it has been used for bonding during dimerization.

A solubility study was conducted on the Dicannabidiol adipate at different pHs. Twelve vials were prepared by placing 1 mg of the dimer in 1 mL of water and ethanol of different pH, from pH 1 to pH 12. It was found that the Dicannabidiol adipate is not soluble in acidic conditions but solubilizes in basic pH. The hypothesis for the dimer being soluble in basic water is due to the bonding of the base with the phenolic oxygen of the dimer, thus producing the salt and easily solubilizing in water. The solubility results suggest that the Dicannabidiol adipate can be used for oral administration to release in the intestines, as well as to intravenous administration. Adipic acid is widely used in food and medicinal applications and is considered compatible and safe in the human body.

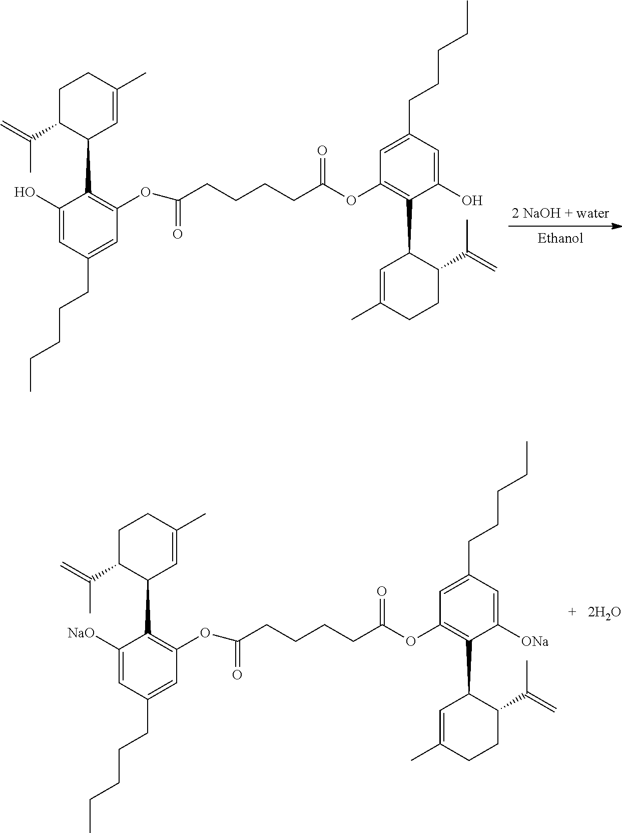

TABLE 1
pH values of the human body

| Body fluid | pH |
|---|---|
| Brain | 7.1 |
| Saliva | 6.0-7.4 |
| Heart | 7.0-7.4 |
| Gastric Secretion | 1.0-3.5 |
| Bile | 7.8 |
| Liver | 7.2 |
| Pancreatic Secretion | 8.0-8.3 |
| Small Intestine Secretion | 7.5-8.0 |
| Urine | 4.5-8.0 |
| Bone | 7.4 |
| Skeletal muscle | 6.9-7.2 |
| Arterial Blood | 7.4-7.45 |
| Venous Blood | 7.3-7.35 |
| Capillary Blood | 7.35-7.4 |

A degradation study was conducted in two parts: thermal degradation and UV light degradation. In the thermal degradation study, the Dicannabidiol adipate was heated to 175° C. in aerobic and anaerobic conditions and the changes were monitored by 1H NMR spectroscopy. In the UV light exposure study, a sample of Dicannabidiol adipate was kept under environmental conditions and UV light for several days and was constantly monitored by 1H NMR spectroscopy. From the results of the two experiments, it was concluded that the Dicannabidiol adipate is stable against the degradation caused from heat and UV exposure. As a result, it does not require expensive or complex storage conditions to maintain its bioactivity, making it suitable for packaging and distribution purposes upon commercialization. Furthermore, as Dicannabidiol adipate is thermally stable, it does not degrade into psychoactive molecules such as delta-9 tetrahydrocannabinol (THC), as cannabidiol (CBD) does. This limits its misuse and abuse. Further, this indicates there will be no need to refrigerate the CBD-adipate dimer drug, and less issues, compared to CBD, with its shipping/handling.

### Example 2. Sulfonation of Dicannabidiol Adipate

Sulfonation of the Dicannabidiol adipate dimer of Example 1 is an approach to increase the water solubility and therefore the bioavailability of cannabinoids or cannabinoid oligomers for pharmaceutical applications. Increasing the bioavailability will allow for cannabinoids as a drug to be more efficiently delivered to the active sites. It is known that some cannabinoids are psychoactive drugs and can bind to receptors in the central nervous system (CNS). Cannabinoids being naturally derived make them a suitable option for advanced biomedical applications. However, their lipophilic nature hinders their direct applications. Hence, to enhance the efficacy of cannabinoids they can be functionalized to be water soluble. Sulfonation using either chlorosulfonic acid, or propane sultone or sulfuryl chloride can be used. Scheme 1 illustrates proposed sulfonation of CBD dimer. Not only dimers, but all cannabinoids, irrespective of the presence of —OH group, can be made water soluble by this method.

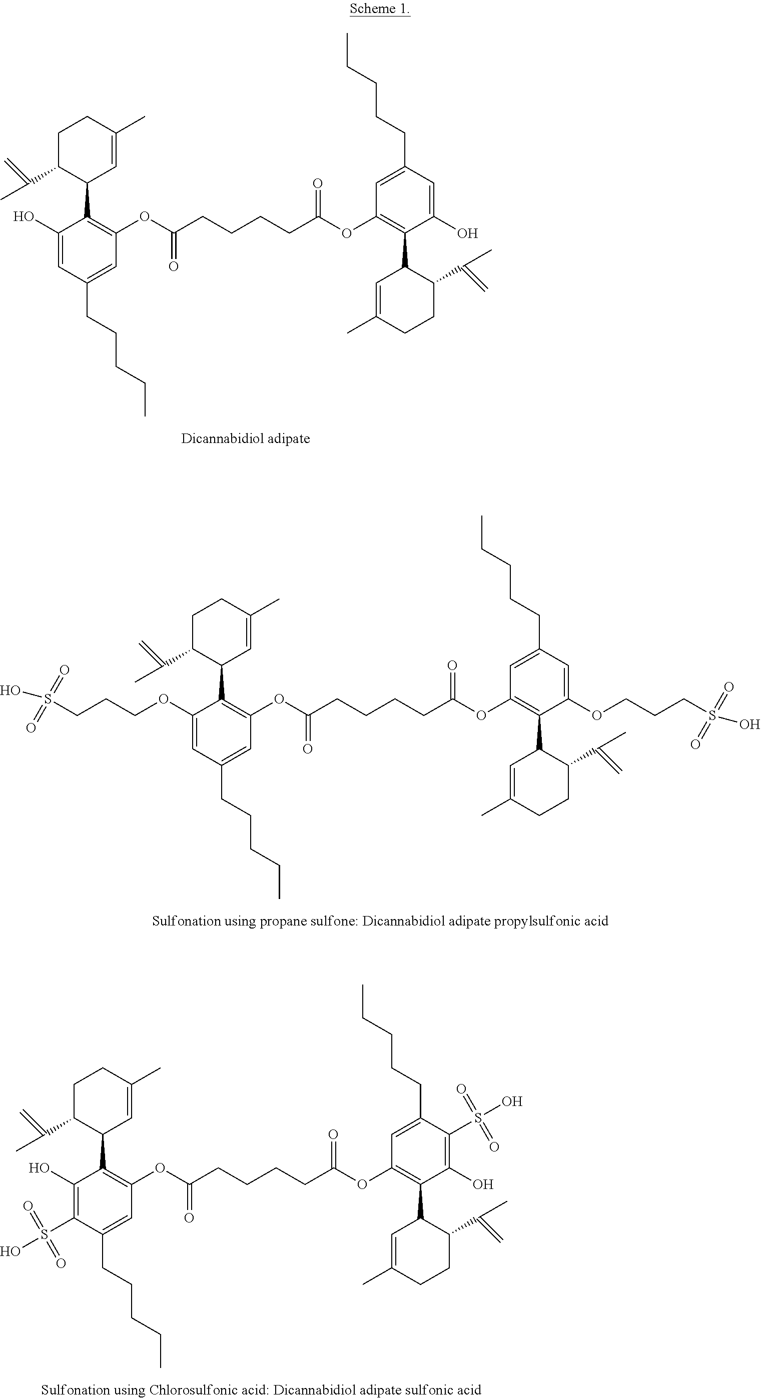

### Example 3. In Vitro Cytocompatibility and Anti-Inflammatory Properties of Dicannabidiol Adipate

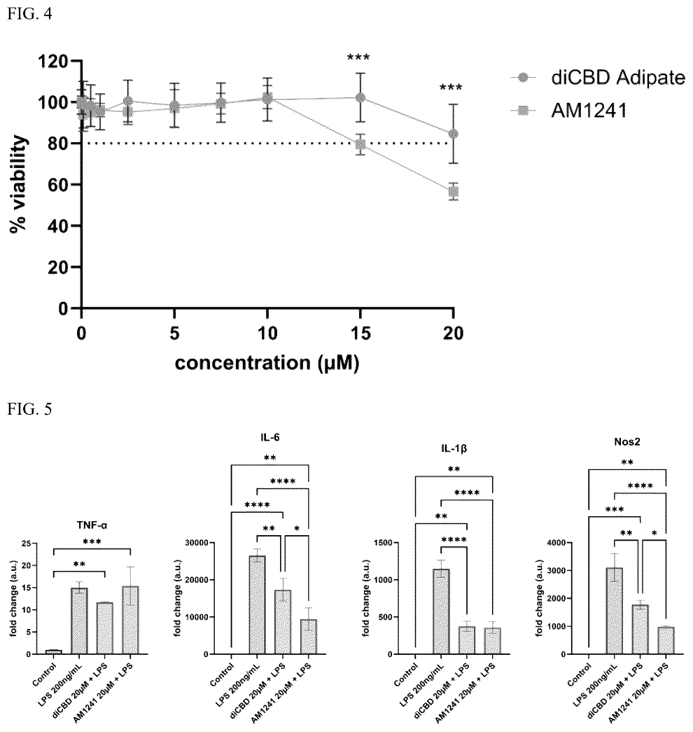

FIG. 4 shows the dose dependent cytotoxicity of Dicannabidiol adipate towards neuronal F11 cells. In vitro, Dicannabidiol adipate showed minimal cytotoxicity, with cell viability remaining above 80% at a concentration of 20 μM. Comparatively, AM1241 (CAS 444912-48-5), a commercially available cannabinoid receptor 2 agonist showed significantly increased cytotoxicity at equivalent concentrations. These data demonstrate the improved cytocompatibility and safety of Dicannabinoid adipate in relation to commercially available cannabinoid receptor modulating drugs such as AM1241.

FIG. 5 shows the anti-inflammatory response of Dicannabidiol adipate and AM1241 towards activated bone marrow macrophages as indicated by the changes in the gene expression of the pro-inflammatory markers TNF-α, IL-6, IL-β and Nos. 2. AM1241 was used as comparison due to its commercial availability. Using equal dosages of 20 μM for both Dicannabidiol adipate and AM1241, the study showed that both molecules exhibited similar anti-inflammatory profiles. Both AM1241 and Dicannabidiol adipate were able to significantly downregulate IL-6, IL-1B, and Nos2 expression. Neither of these drugs showed any significant effect towards TNF-α. Even though AM1241 showed an improved anti-inflammatory effect versus Dicannabidiol adipate in terms of expression of IL-6 and Nos2, the selected dose for Dicannabidiol adipate shows no cytotoxicity as described in FIG. 4.

### Example 4. Preparation of Dicannabidiol Adipate PLGA Microspheres

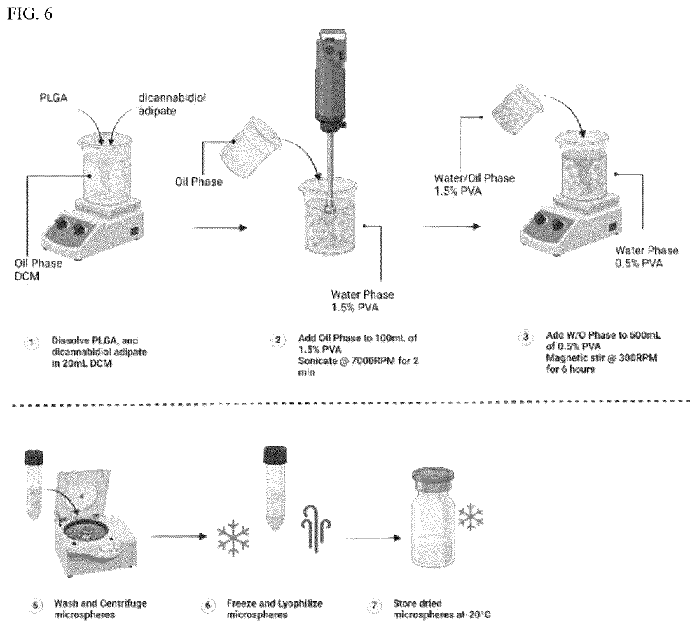

Dicannabidiol adipate loaded microspheres were prepared using the solvent evaporation method using a blend of two different ratios of poly(lactic-co-glycolic) acid (PLGA) (FIG. 6). The two selected ratios of PLGA were: 75:25, and 50:50. These were then weighed and mixed together in a 3:1 ratio of 75:25 PLGA to 50:50 PLGA. For example, for preparing a total of 100 mg of microspheres, 75 mg of 75:25 PLGA was mixed with 25 mg of 50:50 PLGA. Dicannabidiol adipate was incorporated into the microsphere preparation at three different drug-to-total PLGA ratios. The three selected ratios were 1:3, 1:2 and 1:1 Dicannabidiol adipate to total PLGA weight. For example, if using 100 mg of total PLGA, 30 mg, 50 mg, or 100 mg of Dicannabidiol adipate were weighed out and added to the dry PLGA crystals depending on the preparation ratio of interest. Total drug and PLGA was then dissolved in 20 mL of DCM under vortexing. The 20 mL of dissolved diCBD adipate-PLGA-DCM were injected into 100 mL of 1.5% polyvinylalcohol (PVA) using 10 mL syringes with a 19G syringe. The oil-water mixture was then sonicated at 7000 RPM for 2 minutes using a T18 Digital Ultra Turrax sonicator (IKA 0003720001). After sonication, the oil-water mixture was poured into a beaker containing 500 mL of 0.5% PVA and magnetic stirred for 6 hours to evaporate out DCM from the water mixture. After evaporation of the DCM, the microspheres were collected in at least 3 rounds of centrifugation and washing with diH₂O. Microspheres were then frozen overnight at −80° C. and lyophilized for 24 hours. After lyophilization, dried microspheres were stored at −20° C. until further use.

### Characterization of Dicannabidiol Adipate PLGA Microspheres

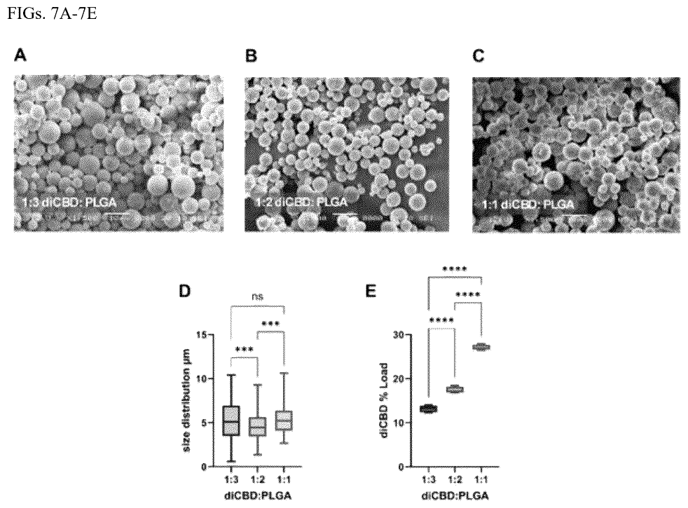

FIGS. 7A-7C show representative SEM images of Dicannabidiol adipate microsphere preparations using three different Dicannabidiol adipate to total PLGA ratios. The microspheres in all three groups showed uniform spherical shapes, but showed some surface roughness and texturing. This surface roughness appeared to increase, as the ratio of Dicannabidiol adipate to PLGA was increased from 1:3, to 1:1. Visual examination of the 1:3 Dicannabidiol adipate to PLGA microspheres using SEM showed that highly spherical and smooth microspheres were generated (FIG. 7A). These microspheres had no large plaques indicating proper encapsulation of Dicannabidiol adipate into the spherical core, instead of surface deposition of Dicannabidiol adipate. The in vitro data presented in FIG. 4 and FIG. 5 showed minimal cytotoxicity of Dicannabidiol adipate up to a tested dose of 20 μM. Dicannabidiol adipate at a 20 μM concentration also showed significant reduction of pro-inflammatory markers such as IL-6, IL-1β and Nos2 (FIG. 5). Based on the low cytotoxicity and the demonstrated anti-inflammatory potency, maximizing the total Dicannabidiol adipate load in the microsphere carrier was desirable. This visual SEM imaging of the 1:3 Dicannabidiol adipate group indicated that further encapsulation of Dicannabidiol adipate in the PLGA microspheres could be possible without having significant surface deposition of the drug. This was done by increasing the ratio of Dicannabidiol adipate in relation to PLGA during the microsphere preparation step as described previously. SEM images showed that as the total dosage of Dicannabidiol adipate was increased (e.g. 1:2 and 1:1 diCBD adipate to PLGA), the surface roughness increased. However, SEM did not show any plaque like formation on the surface of these high dose microspheres (FIG. 7B, FIG. 7C).

The microsphere size analysis revealed that all three preparations were below 10 μm in diameter, regardless of Dicannabidiol adipate to PLGA ratio. Microsphere sizes for each group were within 1 μm of each other, with averages of 5.4 μm (1:3), 4.6 μm (1:2), and 5.4 μm (1:1) (FIG. 7D). The size of the three microspheres groups remain within the desirable range that allows them to be easily injected into a rat knee joint. No significant differences in size were found between the lowest (1:3) and highest (1:1) Dicannabidiol adipate microsphere load groups.

Drug loading revealed a large and significant difference in encapsulated Dicannabidiol adipate based on the preparation ratio selected (FIG. 7E). 1:3 Dicannabidiol adipate microspheres had the lowest Dicannabidiol adipate % load, of approximately 13.2%. This was followed by the 1:2 Dicannabidiol adipate group with 17.4% load. Finally, the 1:1 Dicannabidiol adipate microsphere group had the highest % load measured at approximately 27.1%.

When taking into account the SEM images, and the % load measurements, it can be determined that as more Dicannabidiol adipate was introduced during the microsphere preparation steps, the greater the total amount of encapsulated Dicannabidiol adipate became. The lack of widespread plaque-like surface deposits on the 1:1 Dicannabidiol adipate microspheres suggest that the release profile of Dicannabidiol adipate from these microspheres should be more diffusion and degradation dependent, instead of driven primarily from the solubility of surface deposited Dicannabidiol adipate. Overall, the data here show that the 1:1 Dicannabidiol adipate to PLGA preparation achieves the highest degree of drug loading among the drug to PLGA ratios studied, while maintaining a consistent spherical microsphere morphology, as well as a small enough size that allows for easy injection. Therefore, the 1:1 dicannabidiol adipate to PLGA microsphere preparation was selected for all the following studies.

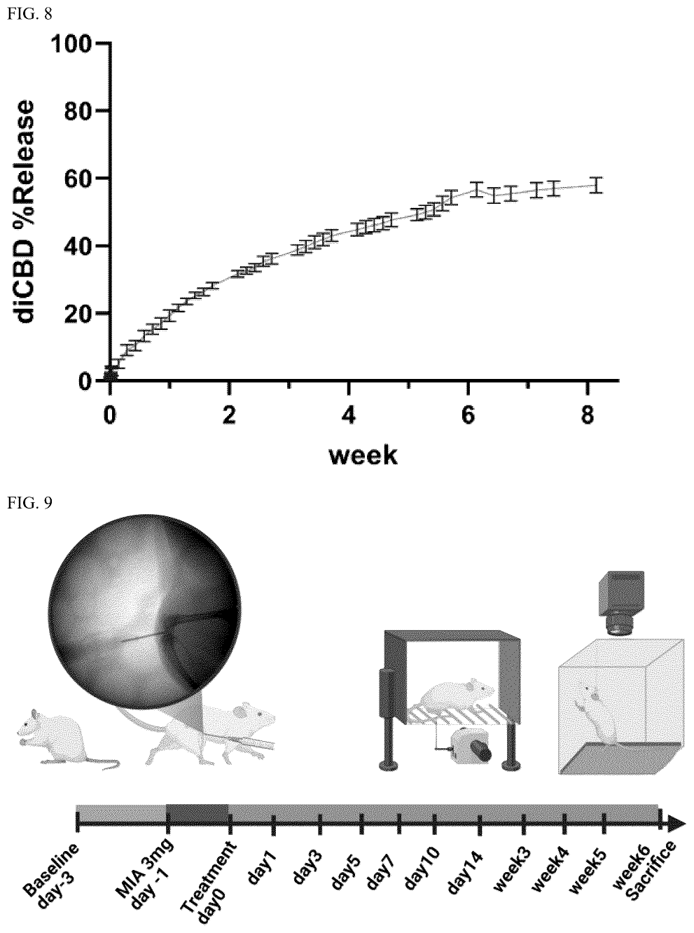

FIG. 8 shows the in vitro release curve of Dicannabidiol adipate from the 1:1 Dicannabidiol adipate-to-PLGA microsphere formulation. The Dicannabidiol adipate microspheres showed a slow and controlled release profile for the entire duration of the study. In the first week, the cumulative release was approximately 19%. By week 4, it had risen to reach approximately 45% Dicannabidiol adipate released. At the week 8 terminal timepoint of the study, almost 60% Dicannabidiol adipate had been released from the microspheres system. The slow and controlled release rate observed in this study supports the observation that the majority of the Dicannabidiol adipate used for microsphere synthesis is encapsulated within the microsphere core, instead of surface deposited. As only 60% of total Dicannabidiol adipate was released within the time course of this study, it is possible that Dicannabidiol adipate may continue to be released beyond the 8-week terminal timepoint that was designated for this in vitro study.

Overall, the Dicannabidiol adipate PLGA microspheres showed enhanced drug loading and a prolonged in vitro release rate. Microsphere size was also successfully kept within the desired parameters for ease of injectability into the osteoarthritis preclinical model.

### Example 5. In Vivo Evaluation of Dicannabidiol Adipate PLGA Microspheres

Based on the results from the in vitro release study, the 1:1 Dicannabidiol adipate to PLGA microsphere formulation was selected for further evaluation in a rat osteoarthritis in vivo model. Briefly, baseline behavioral measurements were quantified prior to induction of osteoarthritis. Monoiodo acetate was used to induce osteoarthritis via intra-articular injection into the rear right knee joint followed 24 hours later by a single intra-articular injection of the designated treatment group (FIG. 9).

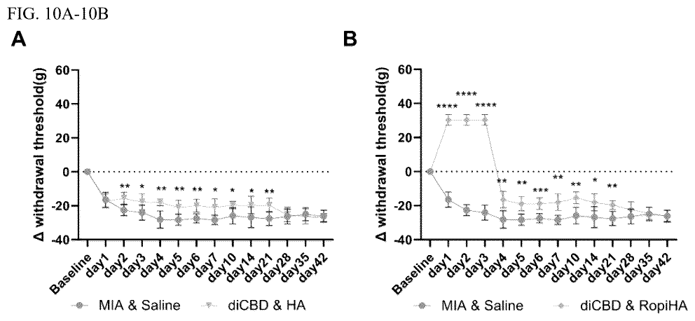

FIG. 10A shows the in vivo analgesic effect of Dicannabidiol adipate microspheres suspended in hyaluronic acid (diCBD) and FIG. 10B shows the combined effect of Dicannabidiol adipate microspheres and Ropivacaine suspended in hyaluronic acid (diCBD and RopiHA). A single intra-articular injection of 3 mg monoiodo acetate (MIA), followed by a vehicle injection of saline 24 hours later induces consistent mechanical sensitivity for the entire duration of the study (FIG. 10A). The single intra-articular injection of the Dicannabidiol adipate microspheres showed similar withdrawal threshold values compared to the MIA and saline treatment group in the first 24 hours post-injection, indicating no analgesic effect was present at the early timepoint. However, from day 2 onwards until day 21 post-injection, withdrawal thresholds in the Dicannabidiol adipate group were increased in comparison to the MIA and saline treatment group indicating that Dicannabidiol adipate was finally being released in sufficient concentrations within the intra-articular space to induce an analgesic effect.

The combined intervention of Dicannabidiol adipate microsphere with the Ropivacaine HA solution showed greater analgesic effects. From day 1 to day 3 post injection, a high degree of hypoalgesia was detected. This hypoalgesic effect was induced by the local anesthetic Ropivacaine. Starting from day 4 onwards, the high degree of analgesia tapered off, but remained statistically significant compared to the MIA and saline treatment group for up to 21 days post injection (FIG. 10B).

These results indicate that a single injection of this novel cannabidiol alternative was sufficient to induce an analgesic effect as measured by both evoked pain assessment methods. Mechanical sensitivity was decreased for up to 21 days when Dicannabidiol adipate microspheres were injected into the intra-articular joint with or without the presence of the local anesthetic Ropivacaine.

ZILRETTA® is the only clinically approved PLGA microsphere formulation for treating knee osteoarthritic pain. It uses PLGA microspheres to locally deliver the corticosteroid triamcinolone acetonide intra-articulably and provides sustained pain relief. While ZILRETTA® demonstrated prolonged pain relief, it does have some limitations.

Size/diameter of ZILRETTA® microspheres: range of 20 μm-100 μm. Average of 40 μm. The preclinical paper where this formulation was studied in osteoarthritic rats does not control microsphere size and any potential cartilage degradation (Kumar et al. 2015). As PLGA is a hard polymer, reduction of microsphere size is desirable when injecting into knee joints. The Dicannabidiol adipate loaded microspheres are an average of 5.4 μm (±2.1 μm), significantly smaller than the reported 40 μm of ZILRETTA® microspheres.

Corticosteroid side effects: while ZILRETTA® can produce pain relief in patients suffering from knee osteoarthritis, long term use of corticosteroids is not recommended as they have been shown to induce chondrocyte toxicity, and cartilage damage. In a chronic disease such as osteoarthritis this is a particular concern. Dicannabidiol adipate showed minimal toxicity in vitro in a dose dependent manner, indicating its safety. Other cannabinoids, such as cannabidiol and cannabigerol have also been shown to reduce osteoarthritic progression in mice, further showing the safety of cannabinoid-based therapies.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. “Or” means “and/or.” The suffix “(s)” as used herein is intended to include both the singular and the plural of the term that it modifies, thereby including one or more of that term (e.g., the film(s) includes one or more films). The terms “including” and “having” are intended to be inclusive such that there may be additional elements other than the listed elements. The term “exemplary” is not intended to be construed as a superlative example but merely one of many possible examples. Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the de-scribed elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and in-stances where it does not. Unless defined otherwise, technical and scientific terms used herein have the same meaning as is commonly understood by one of skill in the art to which this invention belongs.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements de-scribed in an exemplary embodiment without departing from the scope of the various embodiments as set forth in the appended claims.

1. A formulation, comprising:
a microsphere composition; and
an amine/amide anesthetic;
wherein the microsphere composition comprises
a cannabinoid according to formula (I) or (II) 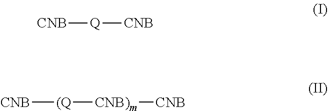
wherein each CNB individually is a cannabinoid moiety; Q is a linking group attached to each cannabinoid moiety independently through an amine, an ether, a thioether, an ester, or an amido group; and m is 1-10; and
a biodegradable polymer.

2. The formulation of claim 1, wherein the amine/amide anesthetic is Articaine, Bupivacaine, Etidocaine, Lidocaine, Mepivacaine, Prilocaine, Procaine, Ropivacaine, or Tetracaine.

3. The formulation of claim 1, wherein the biodegradable polymer is a polylactic acid, a pol (glycolic acid), a poly(lactic-co-glycolic) acid (PLGA), a polycaprolactone, or a polycannabinoid.

4. The formulation of claim 1, wherein the average size of the microsphere is below 40 μm in diameter.

5. The formulation of claim 1, wherein the cannabinoid moiety is derived from
1

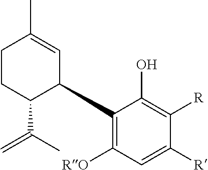

| Sl.N | Molecule Name | R | R′ | R″ |
|---|---|---|---|---|
| la | Cannabidiol | H | C₅H₁₁ | H |
| 1b | Cannabidiolic acid | COOH | C₅H₁₁ | H |
| 1c | Cannabidiol monomethylether | H | C₅H₁₁ | CH₃ |
| 1d | Cannabidibutol | H | C₄H₉ | H |
| le | Cannabidivarine | H | C₃H₇ | H |
| 1f | Cannabidivarinic acid | COOH | C₃H₇ | H |
| 1g | Cannabidiorcol | H | CH₃ | H |
| 1h | Cannabidihexol | H | C₆H₁₃ | H |
| li | Cannabidiphorol | H | C₇H₁₅ | H |

2

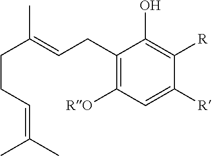

| Sl.N | Molecule Name | R | R′ | R″ |
|---|---|---|---|---|
| 2a | Cannabigerol | H | C5H11 | H |
| 2b | Cannabigerolic acid | COOH | C5H11 | H |
| 2c | Cannabigerol monomethylether | H | C5H11 | CH3 |
| 2d | Monomethylether Cannabigerolic acid | COOH | C5H11 | CH3 |
| 2e | Cannabigerovarine | H | C3H7 | H |
| 2f | Cannabigerovarinic acid | COOH | C3H7 | H |

3

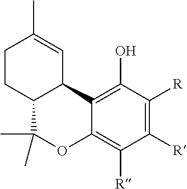

| Sl.No | Molecule Name | R | R′ | R″ |
|---|---|---|---|---|
| 3a | D9-tetrahydrocannabinol | H | C5H11 | H |
| 3b | D9-tetrahydrocannabinolic acid | COOH | C5H11 | H |
| 3c | D9-tetrahydrocannabinolic acid B | H | C5H11 | COOH |
| 3d | D9-tetrahydrocannabinol-C4 | H | C4H9 | H |
| 3e | D9-tetrahydrocannabinolic acid-C4 | COOH | C4H9 | H |
| 3f | D9-tetrahydrocannabivarinic acid | H | C3H7 | H |
| 3g | D9-tetrahydrocannabivarinic acid | COOH | C3H7 | H |
| 3h | D9-tetrahydrocannabiorcol | H | CH3 | H |
| 3i | D9-tetrahydrocannabiorcolic acid | COOH | CH3 | H |
| 3j | D9-tetrahydrocannabinal | CHO | C5H11 | H |
| 3k | D9-tetrahydrocannabiphorol | H | C7H15 | H |
| 3l | D9-tetrahydrocannabihexol | H | C6H13 | H |

4

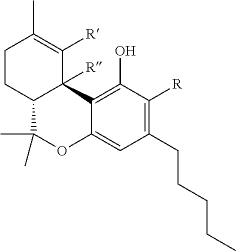

| Sl.No | Molecule Name | R | R′ | R″ |
|---|---|---|---|---|
| 4a | D8-tetrahydrocannabinol | H | H | H |
| 4b | D8-tetrahydrocannabinolic acid | COOH | H | H |
| 4c | 10α-hydroxy-Δ8-tetra-hydrocannabinol | H | α-OH | H |
| 4d | 10β-hydroxy-Δ8-tetra-hydrocannabinol | H | H | β-OH |
| 4e | 10a-α-hydroxy-10-oxo-Δ8- | H | O | OH |
| | tetrahydrocannabinol | | | |

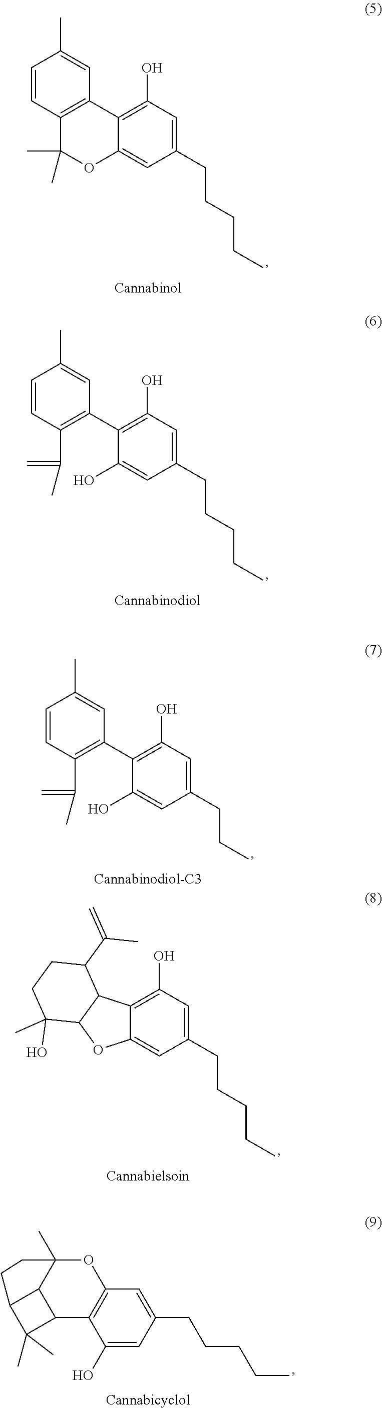

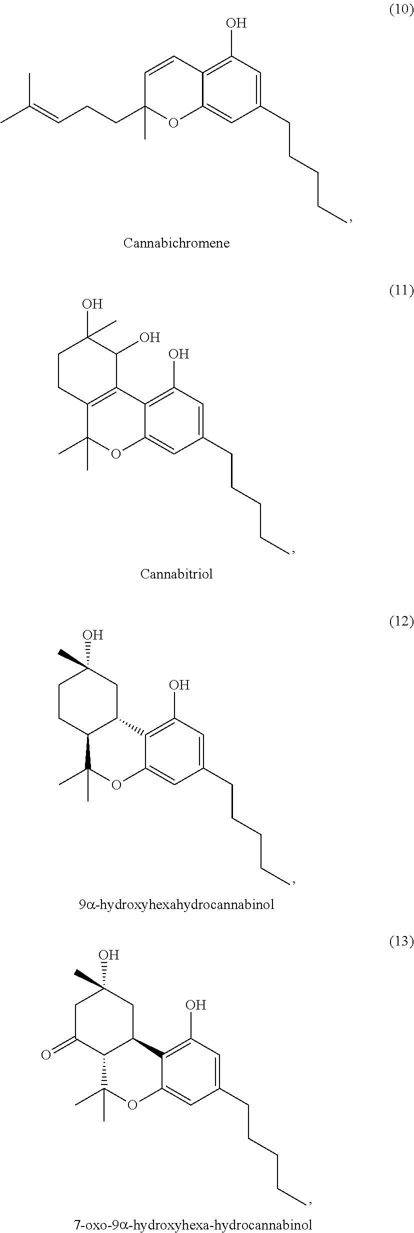

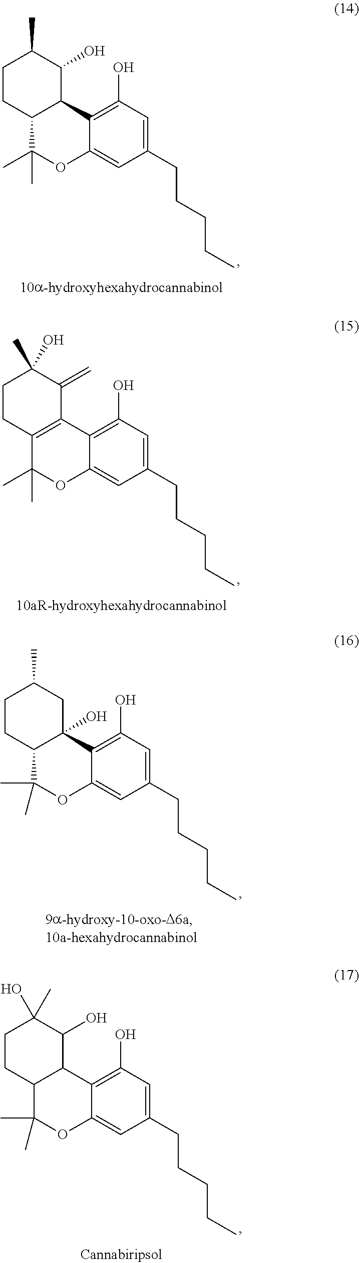

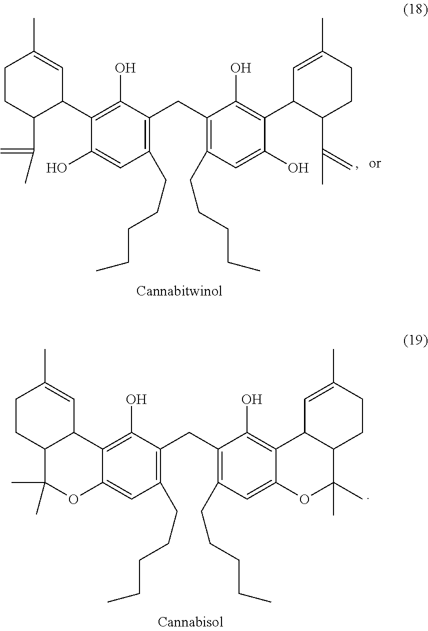

6. The formulation of claim 1, wherein the L linking group is a C₁-C₄₀ alkyl group; an ethylene glycol group (—OCH₂CH₂—); a propylene glycol group; an aryl group; a heteroaryl group; a heterocyclic group; an internally substituted C₂-C₄₀ alkyl group containing 1, 2, or 3 heteroatoms within the carbon chain itself (not at a terminus) wherein the heteroatom is O, S, N, or a combination thereof.

7. The formulation of claim 1, wherein the L linking group is derived from fumaric acid, glutamic acid, maleic acid, malic acid, terephthalic acid, isophthalic acid, naphthalene-2,6-dicarboxylic acid, oxaloacetic acid, phthalic acid, butanedioic acid, oxalic acid, malonic acid, succinic acid, tartaric acid, glutaric acid, adipic acid, pimelic acid (heptanedioic acid), suberic acid (octanedioic acid), azelaic acid (nonanedioic acid), sebacic acid (decanedioic acid), undecanedioic acid, dodecanedioic acid, pyridine-2,6-dicarboxylic acid, 1H-imidazole-4,5-dicarboxylic acid, furan-2,5-dicarboxylic acid, furan-2,3-dicarboxylic acid, thiophene-2,5-dicarboxylic acid, thiophene-2,3-dicarboxylic acid, quinoline-2,4-dicarboxylic acid, cyclohexane-1,4-dicarboxylic acid, cyclopentane-1,3-dicarboxylic acid, cyclobutane-1,3-dicarboxylic acid, or bicyclo[2.2.2]octane-1,4-dicarboxylic acid, or a bifunctional compound such as 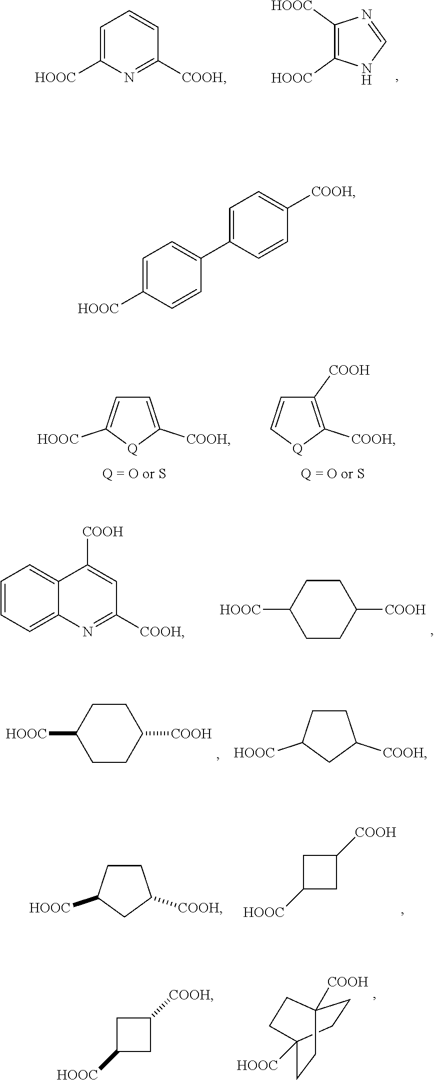
or a derivative thereof.

8. The formulation of claim 1, wherein
the amine/amide anesthetic is Ropivacaine;
the cannabinoid is according to formula (I) 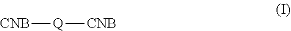
wherein each CNB individually is a cannabidiol moiety or a derivative thereof; Q is a linking group attached to each cannabinoid moiety independently through an ester group and is derived from fumaric acid, glutamic acid, maleic acid, malic acid, terephthalic acid, isophthalic acid, naphthalene-2,6-dicarboxylic acid, oxaloacetic acid, phthalic acid, butanedioic acid, oxalic acid, malonic acid, succinic acid, tartaric acid, glutaric acid, adipic acid, pimelic acid (heptanedioic acid), suberic acid (octanedioic acid), azelaic acid (nonanedioic acid), sebacic acid (decanedioic acid), undecanedioic acid, dodecanedioic acid; and
the biodegradable polymer is a poly(lactic-co-glycolic) acid.

9. The formulation of claim 1, formulated for parenteral administration.

10. A method for the treatment of pain, comprising:
co-administering to a subject in need thereof, a microsphere composition; and
an amine/amide anesthetic;
wherein the microsphere composition comprises
a cannabinoid according to formula (I) or (II) 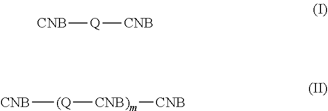
wherein each CNB individually is a cannabinoid moiety; Q is a linking group attached to each cannabinoid moiety independently through an amine, an ether, a thioether, an ester, or an amido group; and m is 1-10; and
a biodegradable polymer.

11. The method of claim 10, wherein the amine/amide anesthetic is Articaine, Bupivacaine, Etidocaine, Lidocaine, Mepivacaine, Prilocaine, Procaine, Ropivacaine, or Tetracaine.

12. The method of claim 10, wherein the microsphere composition and amine/amide anesthetic is administered via parenteral administration.

13. A microsphere composition, comprising:
a cannabinoid according to formula (I) or (II) 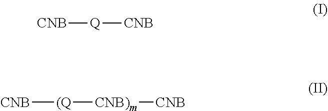
wherein each CNB individually is a cannabinoid moiety; Q is a linking group attached to each cannabinoid moiety independently through an amine, an ether, a thioether, an ester, or an amido group; and m is 1-10; and
a biodegradable polymer.

14. The microsphere composition of claim 13, wherein the biodegradable polymer is a polylactic acid, a pol (glycolic acid), a poly(lactic-co-glycolic) acid (PLGA), a polycaprolactone, or a polycannabinoid.

15. The microsphere composition of claim 13, wherein the average size of the microsphere is below 40 μm in diameter.

16. The microsphere composition of claim 13, wherein the cannabinoid moiety is derived from

| | | | | |
|---|---|---|---|---|
| Sl.N | Molecule Name | R | R′ | R″ |

1

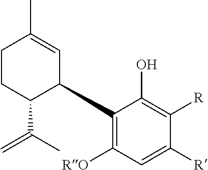

| | | | | |
|---|---|---|---|---|
| la | Cannabidiol | H | C₅H₁₁ | H |
| 1b | Cannabidiolic acid | COOH | C₅H₁₁ | H |
| 1c | Cannabidiol monomethylether | H | C₅H₁₁ | CH₃ |
| 1d | Cannabidibutol | H | C₄H₉ | H |
| le | Cannabidivarine | H | C₃H₇ | H |
| 1f | Cannabidivarinic acid | COOH | C₃H₇ | H |
| 1g | Cannabidiorcol | H | CH₃ | H |
| 1h | Cannabidihexol | H | C₆H₁₃ | H |
| li | Cannabidiphorol | H | C₇H₁₅ | H |

2

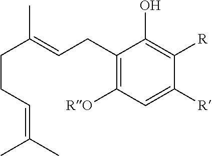

| | | | | |
|---|---|---|---|---|
| 2a | Cannabigerol | H | C5H11 | H |
| 2b | Cannabigerolic acid | COOH | C5H11 | H |
| 2c | Cannabigerol monomethylether | H | C5H11 | CH3 |
| 2d | Monomethylether Cannabigerolic acid | COOH | C5H11 | CH3 |
| 2e | Cannabigerovarine | H | C3H7 | H |
| 2f | Cannabigerovarinic acid | COOH | C3H7 | H |

3

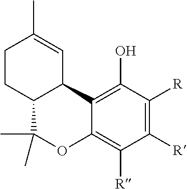

| | | | | |
|---|---|---|---|---|
| 3a | D9-tetrahydrocannabinol | H | C5H11 | H |
| 3b | D9-tetrahydrocannabinolic acid | COOH | C5H11 | H |
| 3c | D9-tetrahydrocannabinolic acid B | H | C5H11 | COOH |
| 3d | D9-tetrahydrocannabinol-C4 | H | C4H9 | H |
| 3e | D9-tetrahydrocannabinolic acid-C4 | COOH | C4H9 | H |
| 3f | D9-tetrahydrocannabivarin | H | C3H7 | H |
| 3g | D9-tetrahydrocannabivarinic acid | COOH | C3H7 | H |
| 3h | D9-tetrahydrocannabiorcol | H | CH3 | H |
| 3i | D9-tetrahydrocannabiorcolic acid | COOH | CH3 | H |
| 3j | D9-tetrahydrocannabinal | CHO | C5H11 | H |
| 3k | D9-tetrahydrocannabiphorol | H | C7H15 | H |
| 31 | D9-tetrahydrocannabihexol | H | C6H13 | H |

4

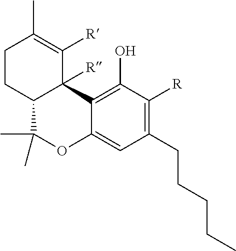

| | | | | |
|---|---|---|---|---|
| 4a | D8-tetrahydrocannabinol | H | H | H |
| 4b | D8-tetrahydrocannabinolic acid | COOH | H | H |
| 4c | 10a-hydroxy-48-tetra-hydrocannabinol | H | α-OH | H |
| 4d | 10ß-hydroxy-48-tetra-hydrocannabinol | H | H | β-OH |
| 4e | 10a-α-hydroxy-10-oxo-Δ8- | H | 0 | OH |
| | tetrahydrocannabinol | | | |

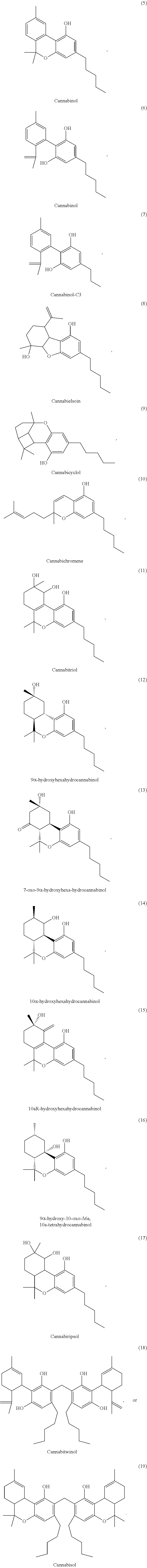

17. The microsphere composition of claim 13, wherein the L linking group is a C₁-C₄₀ alkyl group; an ethylene glycol group (—OCH₂CH₂—); a propylene glycol group; an aryl group; a heteroaryl group; a heterocyclic group; an internally substituted C₂-C₄₀ alkyl group containing 1, 2, or 3 heteroatoms within the carbon chain itself (not at a terminus) wherein the heteroatom is O, S, N, or a combination thereof.

18. The microsphere composition of claim 13, wherein the L linking group is derived from fumaric acid, glutamic acid, maleic acid, malic acid, terephthalic acid, isophthalic acid, naphthalene-2,6-dicarboxylic acid, oxaloacetic acid, phthalic acid, butanedioic acid, oxalic acid, malonic acid, succinic acid, tartaric acid, glutaric acid, adipic acid, pimelic acid (heptanedioic acid), suberic acid (octanedioic acid), azelaic acid (nonanedioic acid), sebacic acid (decanedioic acid), undecanedioic acid, dodecanedioic acid, pyridine-2,6-dicarboxylic acid, 1H-imidazole-4,5-dicarboxylic acid, furan-2,5-dicarboxylic acid, furan-2,3-dicarboxylic acid, thiophene-2,5-dicarboxylic acid, thiophene-2,3-dicarboxylic acid, quinoline-2,4-dicarboxylic acid, cyclohexane-1,4-dicarboxylic acid, cyclopentane-1,3-dicarboxylic acid, cyclobutane-1,3-dicarboxylic acid, or bicyclo[2.2.2]octane-1,4-dicarboxylic acid, or a bifunctional compound such as 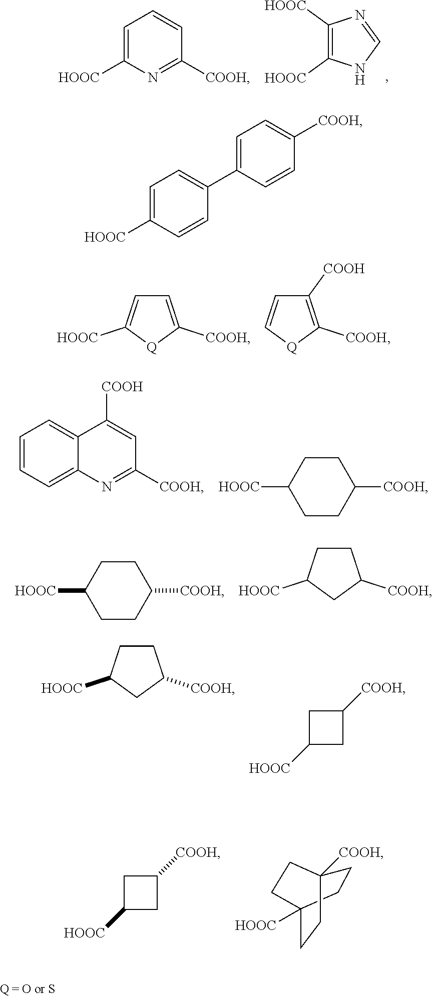
or a derivative thereof.

19. The microsphere composition of claim 13, comprising
the cannabinoid is according to formula (I) 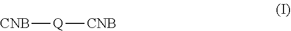
wherein each CNB individually is a cannabidiol moiety or a derivative thereof; Q is a linking group attached to each cannabinoid moiety independently through an ester group and is derived from fumaric acid, glutamic acid, maleic acid, malic acid, terephthalic acid, isophthalic acid, naphthalene-2,6-dicarboxylic acid, oxaloacetic acid, phthalic acid, butanedioic acid, oxalic acid, malonic acid, succinic acid, tartaric acid, glutaric acid, adipic acid, pimelic acid (heptanedioic acid), suberic acid (octanedioic acid), azelaic acid (nonanedioic acid), sebacic acid (decanedioic acid), undecanedioic acid, dodecanedioic acid; and
the biodegradable polymer is a poly(lactic-co-glycolic) acid.
